(19) United States
(12) Patent Application Publication
(10) Pub. No.: US 2021/0106843 A1
(43) Pub. Date: Apr. 15, 2021

(54) INFANT CARE DEVICE INCLUDING LIGHT SHIELDING ENCLOSURE

(71) Applicant: GE Precision Healthcare LLC, Wauwatosa, WI (US)

(72) Inventors: Helge B. Klockow, Waukesha, WI (US); Nagapriya Kavoori Sethumadhavan, Bangalore (IN); Gnanasekar Velusamy, Bangalore (IN); Mohammad Khair, Wauwatosa, WI (US); Thomas Valent, Wauwatosa, WI (US); Steven Falk, Laurel, MD (US)

(73) Assignee: GE Precision Healthcare LLC, Wauwatosa, WI (US)

(21) Appl. No.: 16/598,624

(22) Filed: Oct. 10, 2019

(51) Int. Cl. A61N 5/06; A61G 11/00; G02F 1/163

(52) U.S. Cl. CPC A61N 5/0613; A61G 11/00; G02F 1/163; A61N 2005/0663; A61N 2005/0632; A61N 2005/0638; A61N 2005/0626

(57) ABSTRACT

An infant care device that selectively controls the amount of visible light that is transmitted into the interior of an enclosure while allowing the transmission of light within certain wavelengths. The infant care device includes an enclosure that includes light dimmable technology that adjusts the transparency of the enclosure based upon control signals from a primary controller. The primary controller is able to cycle between day/night on a manual or automatic basis to selectively control the amount of light that reaches the infant patient. The light dimming technology used on the enclosure allows the transmission of light within a phototherapy wavelength spectrum. The controller thus controls the transparency of the enclosure to at least partially reduce or increase the light reaching the infant while allowing phototherapy.

### BACKGROUND

The present disclosure is generally related to the field of infant care. More specifically, the present disclosure is related to a system for controlling the amount of light reaching an infant patient when the infant is within an infant care device, such as an incubator.

Prematurely born infants require specialized treatment and care due to their small size and still-developing organs and physiological systems. Premature infants are very often placed in devices that create a carefully controlled microenvironment around the infant. The microenvironment is designed to provide one or more environmental conditions that are advantageous to the neonate beyond the ambient conditions.

Infant care devices, such as infant warmers, incubators and hybrid units, regulate various different environmental conditions, including humidity, temperature, oxygen content, and many other variables to promote neonate growth. One under-supported variable in the infant care device is light control. Recent studies have shown a correlation between premature infant hospital stay length and light exposure. According to an NIH study by Iris Morag and Anne Ohlsson, cycled light produces significantly higher weight gain and shorter length of stay across a study of premature newborns.

The light shielding products currently in the market to regulate light exposure when the infant is in the incubators, namely hood blankets and room light controls, do not provide patient-specific cycled light. Cycled light within the incubator has the opportunity to impact neonates, their families, and the healthcare industry in an innovative way.

Infant care devices, such as incubators, have multiple modalities which include humidity, noise, and heat control. However, controllable light shielding is still an unassessed issue and an unavailable feature. The present disclosure proposes a controllable light shielding feature that functions to regulate light reaching an infant patient within an incubator.

### SUMMARY

The present disclosure is generally related to the field of infant care. More specifically, the present disclosure is related to a system for controlling the amount of light reaching an infant patient when the infant is within an infant care device, such as an incubator.

An exemplary embodiment of an infant care device includes an infant support platform for supporting an infant patient. The infant support is at least partially surrounded by an enclosure such that a microenvironment can be created within the infant care device. The infant care device is designed such that the transparency of the enclosure can be modified to control the amount of visible light that reaches the infant patient when the infant patient is within the microenvironment. In one exemplary embodiment, the enclosure includes a plurality of walls and a canopy positioned above the wall. Each of the plurality of walls and the canopy includes a light dimming technology that can be operated to control the transparency of the wall or canopy. The light dimming technology is controlled by a primary controller such that the primary controller can selectively control the transparency of the enclosure.

In one embodiment of the disclosure, at least a portion of the canopy is transparent to light within a phototherapy wavelength range such that a phototherapy device can be used with the infant care device even when the transparency of the enclosure is reduced to limit the visible light reaching the infant patient. In one embodiment of the disclosure, the light dimming technology is an electrochromic material that changes transparency depending upon a voltage applied to the material. The primary controller is connected to a variable voltage source that supplies a voltage to the electrochromic material to modify the transparency of the material.

An exemplary embodiment of a method of operating an infant care device positions a light dimming member on an enclosure of the infant care device. A controller is connected to the light dimming member to control the transparency of the light dimming member and thus the amount of visible light that reaches the infant patient. The controller received a desired light cycle that represents the desired times and amounts of light that should reach the infant patient. The controller operates the light dimming member to create periods of darkness and light within the enclosure. The light cycle can be modified based upon a series of parameters that could be patient specific or based on hospital guidelines.

### BRIEF DESCRIPTION OF THE DRAWINGS

The drawings illustrate the best mode presently contemplated of carrying out the disclosure. In the drawings:

FIG. 1 is a perspective view of an exemplary embodiment of an infant care device;

FIG. 2 is a side view of an exemplary phototherapy device utilized with the infant care device;

FIGS. 3A-3F are a series of views showing the transition of the enclosure from fully transparent to fully opaque;

FIG. 4 is a flow diagram illustrating the connections between various components within the infant care device; and

FIG. 5 is an operation flow diagram showing the transitions between different operational states of the controller of the infant care device.

### DETAILED DESCRIPTION OF THE INVENTION

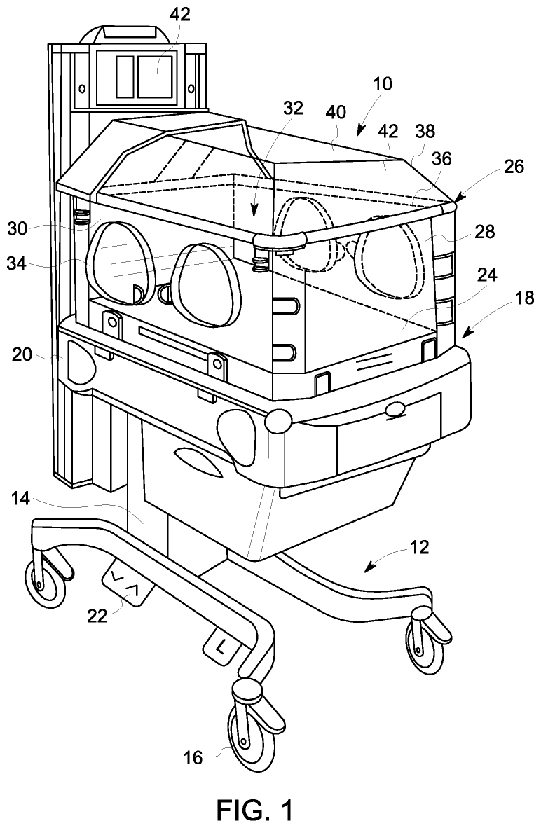

FIG. 1 is a perspective view of an exemplary embodiment of an infant care device 10. The infant care device 10 includes a base 12 which includes a vertical base member 14. The base 12 further includes wheels 16 to facilitate movement of the infant care device 10, for example between use locations, to transport and infant patient, or to move the infant care device to a location for cleaning.

The infant care device includes an infant platform 18 which supports an infant patient in the infant care device 10. The infant platform 18 may be mounted to the base 12 in a cantilevered fashion, for example to a moveable base member 20 which is moveably secured to the vertical base member 14 and is exemplarily moveable in the vertical dimension relative thereto to adjust the height of the infant platform 18 by raising and lowering the moveable base member 20 relative to the vertical base member 14. Exemplarily foot pedals 22 are operable by the user to control the position of the moveable base member 20 and the infant platform 18 to a height preferred by the user.

The infant platform 18 includes a flat, planar surface 24 that underlies and supports the infant patient when the infant patient is positioned within the infant care device 10. In some embodiments, the surface 24 can support a pad or mattress depending upon the age and heath of the infant.

As illustrated in FIG. 1, the infant care device 10 includes an enclosure 26 that creates a microenvironment around the infant when the infant is supported on a mattress or pad positioned on top of the flat, planar surface 24. The enclosure 26 shown in FIG. 1 includes a pair of end walls 28 and a pair of side walls 30. The pair of end walls 28 and side walls 30 define or partially define a microenvironment 32 about the infant patient. In the embodiment shown, the side walls 30 are provided with one or more access ports 34 through which the caregiver can reach the infant patient within the microenvironment 32. The pair of end walls 28 and side walls 30 define a continuous top edge 36 which receives and supports a canopy 38. In the embodiment illustrated, the canopy 38 includes a top wall 40 that is generally planar and is supported by a series of upwardly sloping support walls 42. The support walls 42 each join to the top wall 40 such that the top wall 40 is generally planar and spaced above the support surface 24. Although one possible configuration for the canopy 38 is shown, it should be understood that the canopy 38 could have other configurations while operating within the scope of the present disclosure.

In previously available infant care devices, the entire enclosure 26, including the pair of end walls 28, the side walls 30 along with the canopy 38 were formed from a generally transparent plastic material that allowed ambient light to enter into the microenvironment 32 in an uncontrolled and unregulated manner. In accordance with the present disclosure, a system and method has been developed to automatically or manually control the amount of ambient light that reaches the patient while allowing for selective wavelengths to be blocked and/or transmitted to the infant patient.

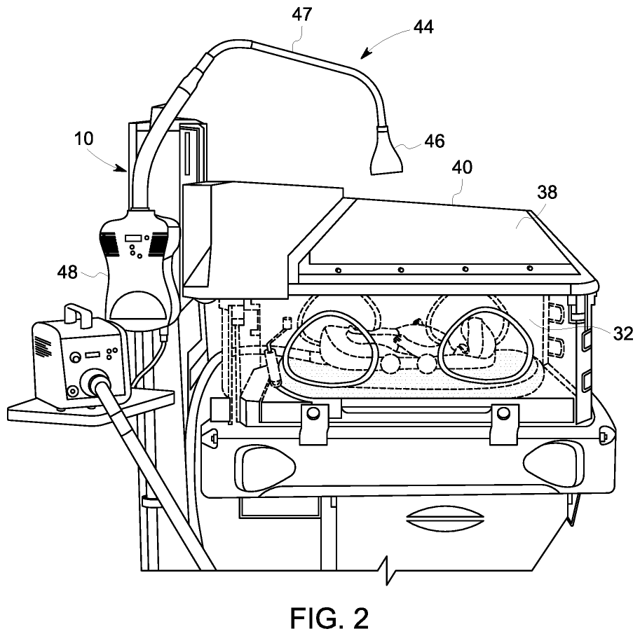

FIG. 2 illustrates a phototherapy device 44 mounted to the infant care device 10 to provide phototherapy to the infant patient when the infant is located within the microenvironment 32 created by the infant care device. In the embodiment shown, the phototherapy device 44 includes an LED light source 46 mounted to a flexible arm 47 that allows the LED light source 46 to be positioned above the top wall 40 of the canopy 38. The LED light source 46 generates a source of blue light in a narrow spectral band. In exemplary embodiments, the LED light source is a blue LED light source that emits light in a narrow spectral band of approximately 430-490 nm, which is the phototherapy wavelength range or band needed to focus on bilirubin's peak absorption wavelength to speed its breakdown. Such light source is used for the effective treatment of hyperbilirubinemia. Thus, for the proper use of the phototherapy device 44 shown, the top wall 40 of the canopy 38 must be transparent to light emitted within the narrow phototherapy wavelength band indicated.

The phototherapy device 44 shown in FIG. 2 includes a control module 48 that controls the intensity of the light emitted as well as the timing of the activation of the LED light 46. The control module 48 can be used as a stand-alone device or can be operatively linked to another controller as will be described in greater detail below.

Although one type of phototherapy device 44 is shown in FIG. 2, it should be understood that other types of phototherapy devices could be utilized while operating within the scope of the present disclosure. As an illustrative example, other types of phototherapy systems are contemplated, such as a bank of LED lights or other types of light sources that are positioned to emit light in a specific phototherapy wavelength band directed onto the infant patient to aid in therapy.

As can be understood in the embodiment of FIGS. 1 and 2, the amount of light reaching the infant patient when the infant patient located within the microenvironment passes through the entire enclosure, including the canopy 38, the end walls 28 and side walls 30. In past systems, to control the amount of light reaching the infant patient, blankets are placed over the canopy 38 to cover the entire enclosure 26 or the lights within the room including the infant care device 10 are controlled. In accordance with the present disclosure, a method and system has been developed and implemented to control the transparency of the enclosure to control the amount of ambient light reaching the infant patient. In addition, a method and system has been developed to control the timing of the transitions between the variable levels of transparency of the enclosure to facilitate different types of therapies. The light shielding system can be automatically controlled by a hospital, caregiver or through manual controls at the infant care device.

In accordance with the present disclosure, the enclosure 26 is formed utilizing one of several different light dimming technologies that control the transparency/opacity of the material used to form the enclosure. Different types of light dimming technologies/techniques are contemplated as being within the scope of the present disclosure. These different light dimming techniques can be either attached to the surface of the transparent end walls 28, side walls 30 and canopy 38 or can be sandwiched in between layers of transparent carrier material in each of these locations. Presently, several different electrically controlled options are contemplated, such as electrochromic devices (ECD), suspended particle devices (SPD), polymer dispersed liquid crystal devices (PDLC), micro blinds and nanocrystals. In each of the electrically controlled options, when an electric signal is applied to the surface including the light dimming technology, the light dimming technology changes the transparency of the material to either increase or decrease the amount of visible light that can pass through the various different surfaces of the enclosure.

In one exemplary embodiment of the present disclosure, the canopy 38, the end walls 28 and the side walls 30 are formed from including an electrochromic (ECD) device whose transparency can be changed by applying different voltage levels to the electrochromic material. In such embodiments, the entire canopy 38, the end walls 28 and the side walls 30 would be covered with a thin film electrochromic material and the thin film electrochromic material is connected to a variable voltage source.

In yet another exemplary embodiment, the canopy could include a PDLC which includes liquid crystal droplets in a polymer matrix. The alignment of the liquid crystals changes when a voltage is applied, thus making the PDLC transparent upon the application of voltage. In such an embodiment, the canopy and walls of the enclosure would be opaque until the application of a required voltage from the variable voltage source.

In yet another contemplated embodiment, a photochromic material could be applied to the enclosure to control the amount of visible light that reaches the infant patient. If a photochromic material were used, the transparency of the photochromic material would be controlled by UV or IR light directed onto the photochromic material from one or more strategically placed diodes.

In a currently preferred embodiment, an electrochromic device is used as the light dimming technology with the enclosure 26. As indicated above, electrochromic devices (ECDs) are composed of multiple material layers that provide a reversible electrochemical redox reaction that changes the transparency of the material based on applied voltage potentials. The outer layers are typically electrodes which are made from two glass or plastic substrates coated with a transparent, electrically polarizable material such as indium tin oxide or ITO. These opposing electrodes allow for the polarization of the two middle layers, the electrolyte and the electrochromic material (ECM), activating the chemical reaction. The electrolyte layer is composed of ions (usually lithium derived) dissolved in a solution. When the ions in the electrolyte solution are polarized by the electrodes, the positive and negative solutes will move toward opposite faces of the layer, allowing them to interact with the ECM. This interaction causes a redox reaction that changes the ECM chemical species to one that attenuates more light as well as expresses a different color visually (i.e. transparent to blue), which is a process called electrochromism. The larger the amount of ions at the ECM surface, the larger the chemical conversion to the more opaque species. Since the amount of reactive ions is controlled by the voltage potential set by the charged electrodes, varying the voltage level will allow for intermediate transparencies within the maximum and minimum levels.

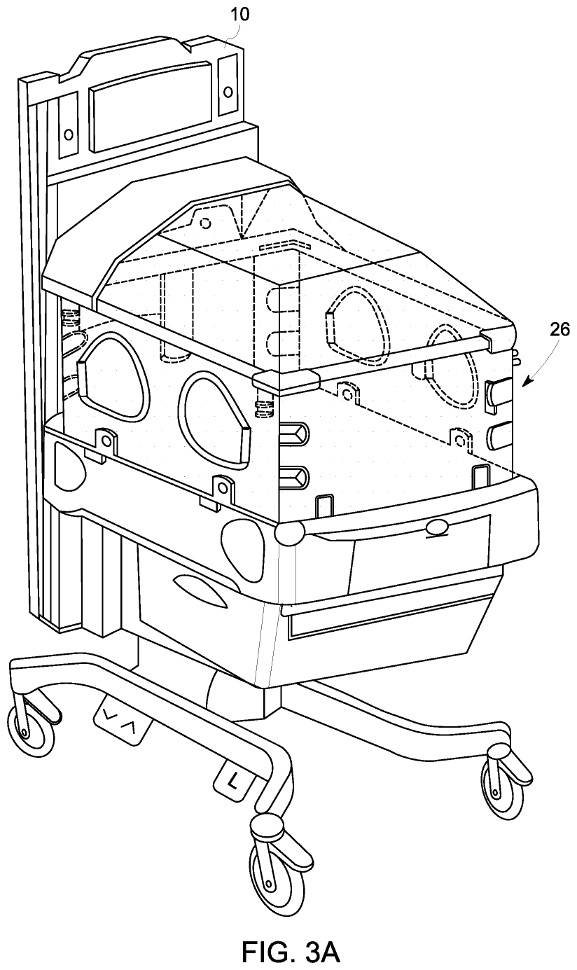

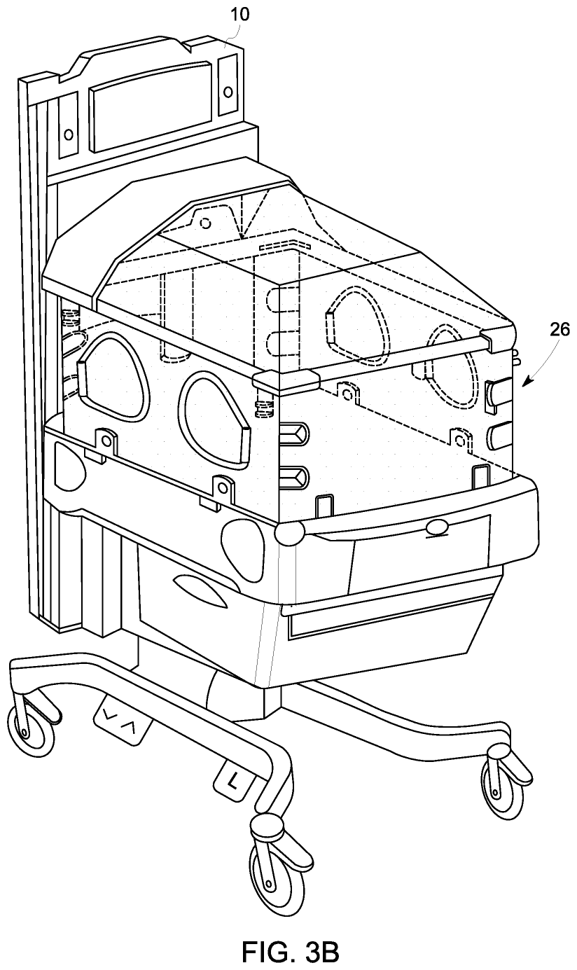

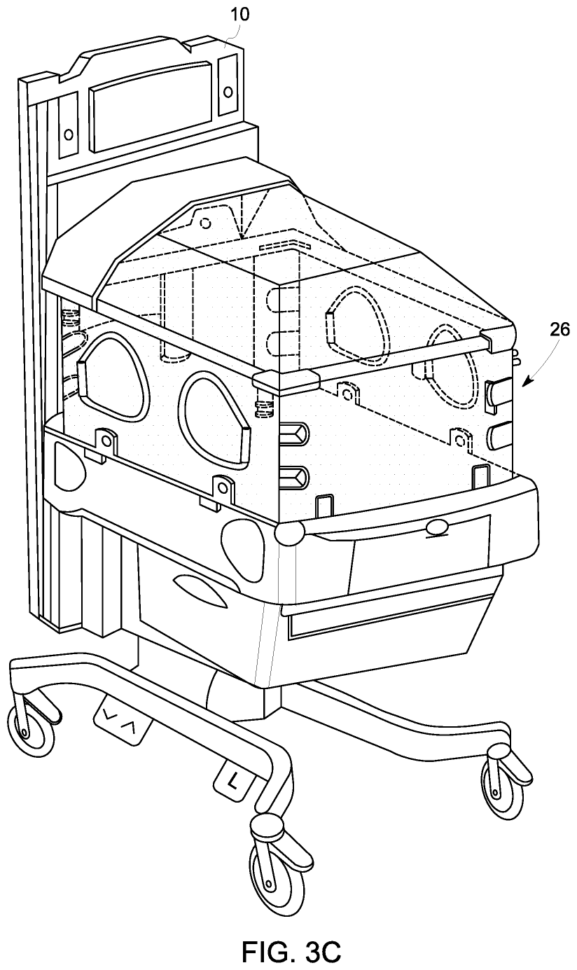

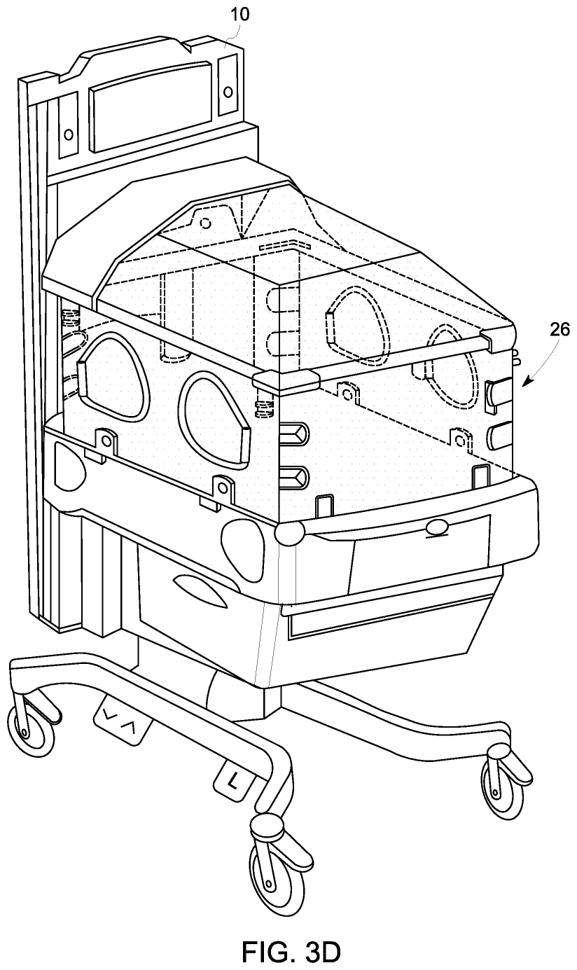

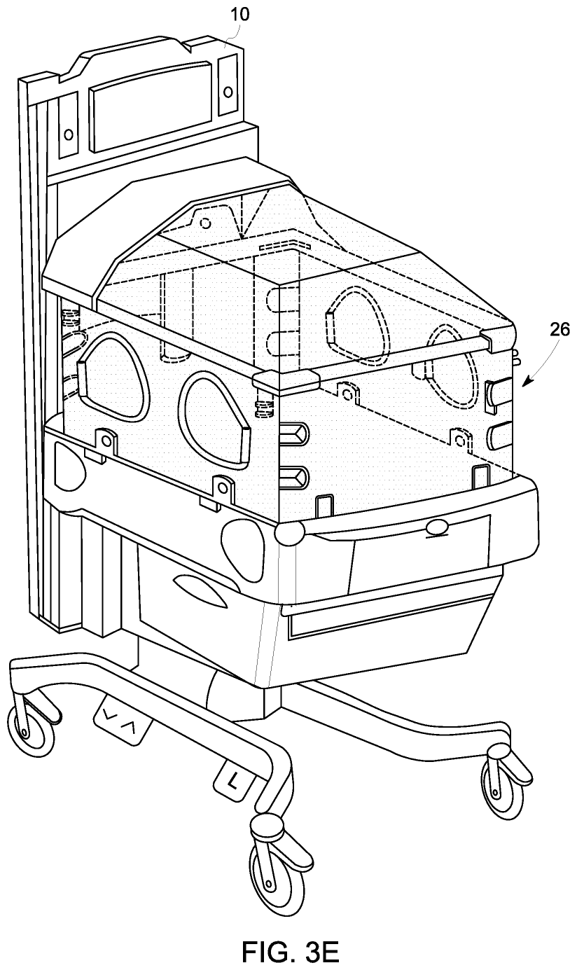

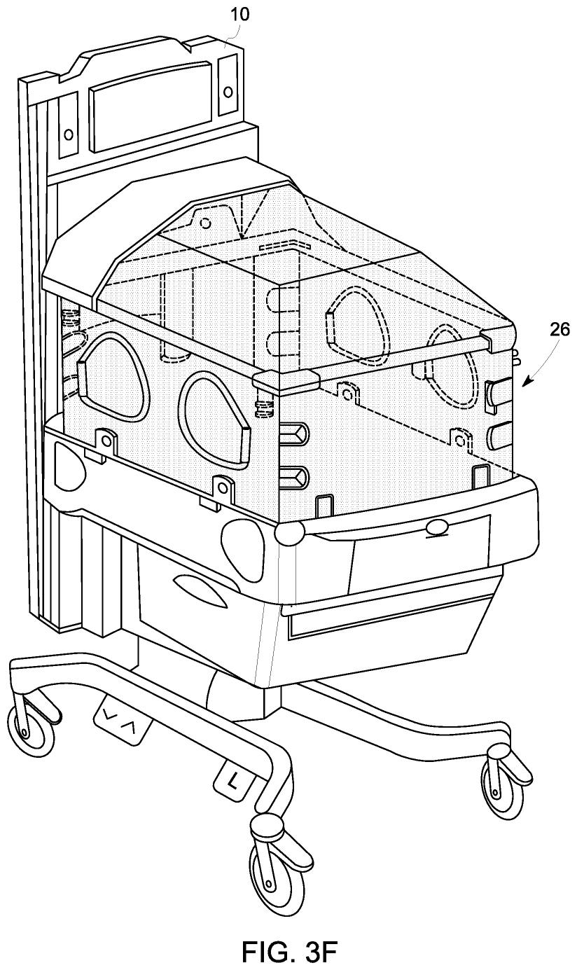

FIGS. 3A-3F illustrates a sequence of views that show the transition of the transparency of the enclosure of the infant care device from the fully transparent state shown in FIG. 3A to the fully opaque condition shown in FIG. 3F. As can be understood in the comparisons of the views shown in FIGS. 3A-3F, the transparency and opacity of the entire enclosure 26 can be controlled by applying variable levels of voltage to the light dimming technology applied to the various surfaces of the otherwise transparent materials used to form the enclosure. In an embodiment in which the light dimming technology utilizes an electrochromic device, the level of transparency can range from 100% shown in FIG. 3A to close to 0% shown in the view of FIG. 3F through the application of different voltage levels to the electrochromic devices applied to the material.

In the embodiment shown in FIGS. 3A-3F, the transparency of the canopy 38, the end walls 28 and the side walls 30 change in synchronization with each other. However, it is contemplated that the control system of the present disclosure could control the transparency of the canopy 38 separately from the transparency of the end walls 28 and separately from the transparency of the side walls 30. In such an embodiment, the canopy 38 could transition to the fully opaque condition shown in the view of FIG. 3F while the end walls and side walls would remain transparent such as shown in the view of FIG. 3A.

As described above with reference to FIG. 2, the infant care device is very often used with a phototherapy device 44 that include an LED light source 46 that generates a source of blue light in a narrow spectral band. In an exemplary embodiment, the LED light source is a blue LED light source that emits light in a narrow spectral band of approximately 430-490 nm. In order for the phototherapy device to be usable with the enclosure that includes the light dimming technology, the light dimming technology must be transparent to the phototherapy light in the phototherapy wavelength spectrum. Thus, the light dimming technology must be entirely transparent to the light emitted within the narrow phototherapy wavelength band or must include a least a section that is transparent and is aligned with the light source of the phototherapy device.

In one exemplary embodiment, the light dimming technology includes electrochromic material that is selectively transparent to light in the visible light spectrum and is fully transparent to light in the phototherapy wavelength range. Thus, even as the transparency of the material to visible light changes, the material allows the phototherapy light to pass thought and be received by the infant patient.

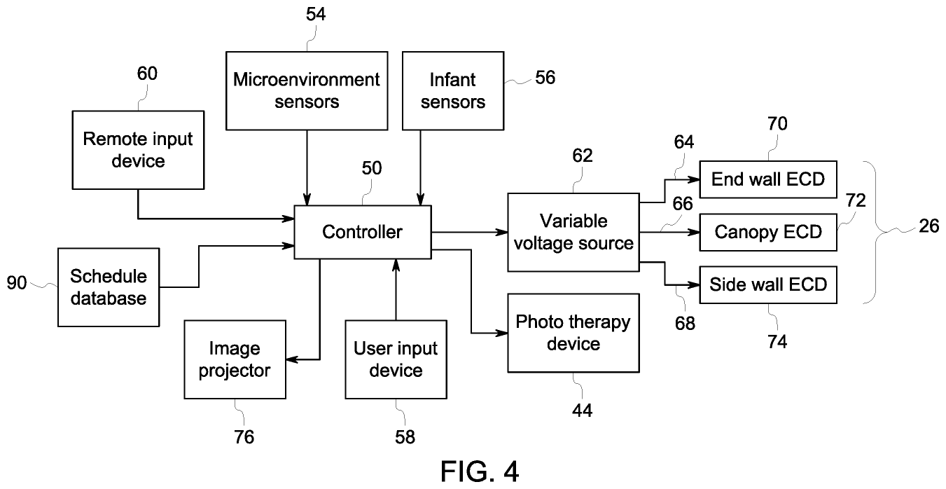

FIG. 4 illustrates one embodiment of a control system utilized in accordance with an exemplary embodiment of the present disclosure. As shown in FIG. 4, the infant care device 10 includes a primary controller 50 that is normally operable to control the operation of the incubator environmental devices 52. These environmental devices 52 can include a heater or cooler to control the temperature within the microenvironment, a humidity source or dehumidifier to control the humidity within the microenvironment as well as other controls that are needed to generally operate the infant care device in a known manner. In order to control these devices, the primary controller 50 receives input from microenvironment sensors 54, which can include temperature sensors located within the microenvironment, humidity sensors, light sensors and the like. Further, the primary controller 50 is connected to a series of infant sensors 56 that provide information to the controller 50 about the current physiological status of the input. This information can be heartrate, skin temperature sensors, SPO2 levels, ECG information, patient weight, patient location or any other type of information that may be desired and is obtained directly from the infant when the infant is located within the infant care device.

A user input device 58 is typically positioned somewhere on or around the infant care device and allows a user to input information into the primary controller 50 from a location at or near the infant care device. In addition, a remote input device 60 can be connected to the primary controller 50 through either a hardwire connection or a wireless connection to allow monitoring and control of the infant care device from a remote location, such as at a nurses' station or other monitoring location.

In accordance with the exemplary embodiment shown in FIG. 4, a phototherapy device 44 is operably connected to the primary controller 50 such that the primary controller 50 can control the operation of the phototherapy device 44. In the embodiment previously described, the phototherapy device can include a separate operating controller that is connected to the primary controller 50 such that the primary controller 50 can monitor and control the operation of the phototherapy device 44.

In accordance with the system of the present disclosure, the primary controller 50 is further connected to a variable voltage source 62. The variable voltage source 62 can be controlled to output one or more different voltage levels along the voltage control lines 64, 66 and 68. Although multiple voltage control lines 64, 66 and 68 are shown in the embodiment, it is contemplated that in an exemplary embodiment, the variable voltage source 62 could include a single voltage output line depending upon the configuration and operation of the light dimming technology used in connection with the infant care device. In other embodiments, the variable voltage source 62 could be replaced by light emitting devices that would be used to control the operation of a photochromic device.

In the embodiment shown in FIG. 4, the voltage control line 64 is operatively connected to the end wall ECD 70. The voltage control line 66 is operatively connected to the canopy ECD 72 while the voltage control line 68 is operatively connected to the side wall ECD 74. In the control embodiment shown in FIG. 4, the primary controller 50 is thus able to independently control the operation and transparency of the three separate ECDs 70, 72, and 74. However, in other alternate contemplated embodiments, the three ECDs shown in FIG. 4 could be combined and the control would be common for each of the three electrochromic devices. In such embodiment, the variable voltage source 62 would generate a single output voltage that would control the ECD attached to the end walls 28, the side walls 30 and the canopy 38. However, the distributed control shown in FIG. 4 allows for further flexibility in controlling the transparency of each of the separate portions of the enclosure 26 as described.

In a contemplated alternate embodiment, the system shown in FIG. 4 can include an optional image projector 76 that is controlled by the primary controller 50. The image projector 76 would be located within the microenvironment created by the enclosure. When the primary controller reduces the transparency of the walls and the canopy 26, the primary controller 50 can selectively operate the image projector 76 located within the enclosure to project some type of image onto any one of the inside surfaces of the enclosure. The projected image could the face of the mother to increase bonding between the mother/father and the infant patient during treatment. Such embodiment would be possible when utilizing an electrochromic device (ECD) or utilizing PDLC technology that, when activated, turns the clear, transparent surface of the enclosure milky white which can be used as a screen for projecting images. The image projector 76 could be an add-on component or utilized when desired with a specific infant patient.

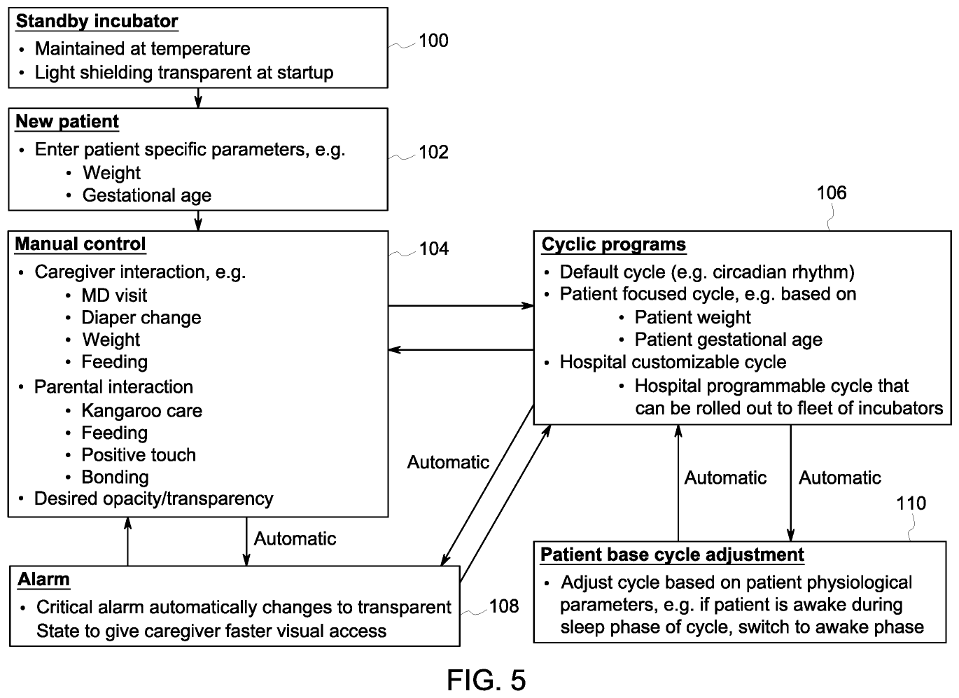

FIG. 5 illustrates general operating procedures and methods in accordance with one exemplary embodiment of the present disclosure in which light dimming technology is incorporated into the infant care device. In step 100 shown in FIG. 5, the infant care device (incubator) is at a standby state before an infant patient is placed within the enclosure. In the standby state, the incubator is maintained at a standby temperature and the light shielding/dimming technology is set to fully transparent. It is desirable that the enclosure is fully transparent in the standby state such that a caregiver and personnel around the incubator can see fully within the enclosure to determine what equipment, sensors, bedding and other components may be located within the enclosure.

When a new infant patient is placed within the incubator, the primary controller enters step 102 in which information about the infant patient is entered into the primary controller. This information can be entered utilizing a remote input device or an input device directly connected to the primary controller at the incubator. Relevant information about the infant patient, such as the weight, age, blood oxygen levels or any other information that may be relevant to maintaining the health of the infant patient when the infant patient is received within the microenvironment created by the incubator.

After the new infant patient has been placed into the incubator, the controller enters into a series of operational sequences and steps that are generally illustrated by the manual control block 104, cycle program block 106, alarm block 108 and patient cycle adjustments 110. It should be understood that the primary controller operates between each of the blocks shown in FIG. 5 and is not required to remain in any one of the operational block. The transition between the operational blocks is controlled by parameters entered into the controller, measurements made from the infant patient or based upon a scheduled set utilizing the control characteristics of the primary controller 50.

In block 104, the primary controller can receive different types of information from a caregiver or any other person located within physical proximity to the user input device 58 shown and described in FIG. 4. As an illustrative example, the caregiver can enter information related to a manual weight reading of the infant, the time of the last feeding, the time of the last diaper change and the time of the last doctor visit. In addition, manual information can be entered about the parental interaction with the infant, such as feeding times, positive touch therapy and other information related to the bonding between the infant patient and the parents. Further, the manual user input device can be used to control and change the opacity/transparency of the enclosure. As an example, if the transparency of the enclosure is set close to 0% and the parents wish to view their infant patient within the enclosure, a caregiver or the parent can manually increase the transparency through manual controls on the user input device. In this manner, the caregiver or parent can adjust the operation of various different parameters and enter information into the primary controller utilizing the user input device 58.

Block 106 identifies various different types of cyclic programs that can be carried out by the primary controller 50 of the present disclosure. Although example programs are shown in block 106, it should be understood that various different programs could be utilized and that the programs specified can be modified/adjusted based upon the infant patient.

The first type of cyclic program shown in block 106 is a default cycle that is meant to simulate a day/night cycle for the infant patient. In such cycle, the transparency of the enclosure will change to simulate the transition between day/night, thereby creating a day/night cycle for the patient. Such cycling will help assimilate the infant patient to the day/night cycle, especially when the ambient light of the room in which the infant care device is located is lit nearly 24 hours a day. Since the primary controller can automatically control the transparency of the enclosure, the primary controller can automatically create a day/night cycle for the infant patient which can be pre-programmed. Additionally, the day/night cycle durations can be adjusted by caregivers to further match the infant patient.

As further indicated in block 106, the default cycle can be modified to be patient focused and based upon the weight of the patient and the gestational age of the patient. In this manner, the caregiver can modify the cycle of day/night or provide periods of darkness for the infant depending upon infant related parameters.

As described previously, the primary controller 50 shown in FIG. 4 is connected to the remote input device as well as a schedule database 90 such that a hospital can roll out programmable cycles that can be utilized with a fleet of incubators. The primary controller 50 can then control the transparency of the enclosure through the control of the variable voltage source 62.

As illustrated in FIG. 5, the primary controller can transition between the manual control and cycle programs in an automatic or manual fashion.

In block 108, if a critical alarm is generated at the infant care device, such as based upon monitor parameters from the infant sensors 56 shown in FIG. 4, the primary controller can transition into an alarm state in which the primary controller controls the operation of the variable voltage source to render each of the walls and the canopy of the enclosure fully transparent. Transitioning each of the walls and canopy to the fully transparent state allows for a caregiver to more quickly visually assess the state of the infant patient rather than having to first cause the transformation of the enclosure from a very low transparency to a high transparency. The transition to the alarm state is automatic based upon any one of multiple parameters that could be selected by the caregiver or the hospital.

In block 110, the cycle set in block 106 can be modified based upon monitored patient parameters, such as from the infant sensors 56 shown in FIG. 4. As an example, the day/night cycle can be adjusted if the patient awakens during the sleep phase of the cycle to simulate daytime for the infant patient. Once again, the cycle adjustments shown in step 110 can be adjusted by the caregiver or by a global change based upon hospital requirements. As an illustrative example, the primary controller can control the transparency of the enclosure based upon physiological parameters such as respiration rate, depth of breath, heartrate, body movement, rapid eye movement, SPO2 concentration, ECG measurements, EEG measurements, temperature, monitored facial expressions or other patient parameters. Besides the patient parameters, other states can be used to adjust the cycle, such as light sensors that determine whether there is ambient light in the room where the incubator is located. In each case, the cycle set in block 106 can be adjusted based upon monitored parameters to further tailor the day/night cycles to the infant patient and the surrounding environment.

The infant care device 10 further includes a user input device 42, which in an exemplary embodiment is a touch sensitive graphical display that is exemplarily used to present both patient as well as operational information to a clinician. The user input device 42 further is operable to receive user inputs from an operating clinician or technician including, but not limited to user inputs regarding the operation and use of the infant care device 10. In embodiments, this may include providing an on/off switch for the infant care device 10. In other embodiments, such a power switch may be provided as a physical switch elsewhere on the infant care device 10. The infant care device 10 further includes a power cord 44 that terminates in a plug 46 which is configured to be operatively connected to an outlet or other external electrical power source configured, for example to provide mains electricity to the infant care device 10.

I claim:

1. An infant care device for use with an infant, comprising:
an infant support;
an enclosure positioned at least partially around the infant support to create a microenvironment within the infant care device,
wherein the transparency of the enclosure to visible light can be modified to control the amount of visible light reaching the infant when the infant is within the infant care device.

2. The infant care device of claim 1 further comprising a controller operable to selectively control the transparency of the enclosure.

3. The infant care device of claim 2 wherein the enclosure includes a plurality of walls and a canopy supported above the plurality of walls.

4. The infant care device of claim 3 wherein the transparency of each of the plurality of walls and the canopy is controlled separately by the controller.

5. The infant care device of claim 3 wherein at least a portion of the canopy is transparent to light within a phototherapy wavelength range.

6. The infant care device of claim 5 wherein the phototherapy wavelength range is 430-490 nm.

7. The infant care device of claim 3 wherein each of the plurality walls and the canopy includes a electrochromic device connected to a voltage source, wherein the controller controls the voltage applied to the electrochromic device to control the transparency of the walls and the canopy to visible light.

8. The infant care device of claim 1 further comprising an image projector located within the infant care device and operable to project an image onto an inner surface of the enclosure when the enclosure is at least partially opaque.

9. An infant care device for use with an infant, comprising:
an infant support;
an enclosure positioned to surround the infant support to create a microenvironment within the infant care device, the enclosure including a plurality side walls, a plurality of end walls and a canopy;
a light dimming member associated with each of the side walls, each of the end walls and the canopy, wherein a level of transparency of each of the end walls, the side walls and the canopy can be modified to control the amount of visible light reaching the infant when the infant is within the infant care device; and
a controller operatively connected to each of the light dimming members to selectively control the transparency of the enclosure.

10. The infant care device of claim 9 wherein the controller operates to control the level of transparency of the enclosure based on a pre-programmed cycle.

11. The infant care device of claim 9 wherein the controller operates to make the enclosure transparent to visible light upon receiving an alarm condition.

12. The infant care device of claim 9 further comprising a user input device connected to the controller, wherein the user input device is operable to manually control the level of transparency of the enclosure.

13. The infant care device of claim 9 wherein at least the canopy is transparent to light in a phototherapy wavelength range.

14. The infant care device of claim 13 wherein the phototherapy wavelength range is 430-490 nm.

15. The infant care device of claim 9 wherein the controller operates to control the level of transparency based on one or more physiological parameters received from the infant.

16. The infant care device of claim 9 further comprising an image projector located within the infant care device and operable to project an image onto an inner surface of the enclosure.

17. The infant care device of claim 10 wherein the pre-programmed cycle simulates a circadian rhythm.

18. A method of controlling the amount of visible light reaching an infant patient positioned within an infant care device includes a support platform and an enclosure located to surround the support platform, the method comprising the steps of:
positioning a light dimming member on the enclosure, wherein the light dimming member is operable to selectively modify the transparency of the enclosure to visible light;
receiving a desired light cycle in a controller of infant care device, wherein the light cycle includes desired periods of darkness and light for the infant patient;
operating the light dimming member to create the periods of darkness and light within the enclosure.

19. The method of claim 18 wherein the controller causes the light dimming member to transition to fully transparent upon receiving an alarm condition.

20. The method of claim 18 wherein the light dimming member is controlled by a voltage and the controller is operable to control the operation of a variable voltage source.
